(19) United States
(12) Patent Application Publication
(10) Pub. No.: US 2026/0030994 A1
(43) Pub. Date: Jan. 29, 2026

(54) Systems and Methods for Cognitive Assessment and Training, and Cognitive Decline Remediation

(71) Applicant: The Regents of the University of California, Oakland, CA (US)

(72) Inventor: Ladan Shams, Los Angeles, CA (US)

(73) Assignee: The Regents of the University of California, Oakland, CA (US)

(21) Appl. No.: 19/279,880

(22) Filed: Jul. 24, 2025

Related U.S. Application Data
(60) Provisional application No. 63/675,150, filed on Jul. 24, 2024.

(51) Int. Cl. G09B 7/02; A61B 5/16; G16H 20/70; G16H 50/20

(52) U.S. Cl. CPC G09B 7/02; A61B 5/165; G16H 20/70; G16H 50/20

(57) ABSTRACT

One embodiment includes a method for cognitive assessment is provided. The method comprises presenting, using an assessment device, one or more tasks to a subject, wherein the one or more tasks include a time series of visual stimuli, audio stimuli, or both visual and audio stimuli, wherein the visual stimuli comprises flashes of light and the audio stimuli comprises short audio tones. The method further comprises receiving, via the assessment device, response data from the subject for each of the one or more tasks; calculating, using one or more processors, performance-related parameters based on the response data, wherein the performance-related parameters include timing data; analyzing, using the one or more processors, the subject's performance as a combination of performance-related parameters selected across the one or more tasks presented to the subject; and generating, using the one or more processors, a cognitive measurement of the subject based on the analyzed performance.

### CROSS-REFERENCE TO RELATED APPLICATIONS

The current application claims the benefit of and priority under 35 U.S.C. § 119 (c) to U.S. Provisional Patent Application No. 63/675,150, entitled “Systems and Methods for Cognitive Assessment and Training, and Cognitive Decline Remediation” filed Jul. 24, 2024, the disclosures of which are hereby incorporated by reference in their entirety for all purposes.

### FIELD OF INVENTION

The present disclosure relates to behavioral methods of cognitive assessment and training, and more particularly to systems and methods for assessing, tracking, improving, and rehabilitating memory and cognition using non-invasive behavioral tasks.

### BACKGROUND

Cognitive ability describes a person's capacity to learn, adapt, solve problems, understand relevant instructions, reason, and plan. Current cognitive assessment tests, such as those used for diagnosing mild cognitive impairment, early onset dementia, Alzheimer's disease, and numerous other degenerative or mental conditions, generally require having a patient visit a doctor, answer test questions, and/or perform various tasks whereby a doctor can then make an assessment regarding the existence and severity of an impairment. Likewise, most measures to prevent or remedy cognitive decline have generally been primarily directed towards using prescribed medications and/or recommending lifestyle changes.

In recent years, computerized cognitive training approaches have gained attention by demonstrating that simple games and exercises can improve an individual's performance in computerized cognitive tasks. However, these approaches have faced challenges in delivering meaningful real-world solutions. One issue is that cognitive training often requires extensive daily practice on the same tasks, and it remains unclear if the gained improvements are long-lasting. Additionally, even when improvement in a specific task is achieved, the benefits have generally been limited to the trained computerized tasks and do not generalize to other tasks or daily life functions.

In daily life, humans are typically exposed to visual and auditory stimuli in their environment, and the brain processes this multisensory input to understand the surroundings and act accordingly. This involves timely, accurate, and precise processing of sound and light. When both auditory and visual information are concurrently available and stem from the same object or event, the brain also combines the information provided from the two senses, a process known as multisensory integration.

Research in the past two decades has established that crossmodal interactions in perceptual processing are prevalent across various tasks, domains, and modalities, as well as across different developmental stages in an individual's life. Integration of signals across the senses is key to perceiving the world accurately and precisely. For example, estimating the location or speed of a car involves integrating the information provided by vision and hearing. Multisensory inference and integration occur continuously and are central to human perception.

Higher-level cognitive functions, such as memory, reading, language, and social skills, rely critically on accurate sensory processing of one's environment and proper integration of information across the senses. Therefore, a deficit or anomaly in unisensory and/or multisensory processing can potentially have wide-ranging consequences for cognitive function.

There is a growing interest in developing more effective and generalizable methods for cognitive assessment, training, and remediation. Approaches that can provide accurate evaluation of cognitive abilities, offer engaging and efficient training protocols, and potentially address cognitive decline through non-invasive behavioral methods are of particular interest in the field.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

In one embodiment, a method for cognitive assessment is provided. The method comprises presenting, using an assessment device, one or more tasks to a subject, wherein the one or more tasks include a time series of visual stimuli, audio stimuli, or both visual and audio stimuli, wherein the visual stimuli comprises flashes of light and the audio stimuli comprises short audio tones, wherein the short audio tones comprises noise bursts. The method further comprises receiving, via the assessment device, response data from the subject for each of the one or more tasks; calculating, using one or more processors, performance-related parameters based on the response data, wherein the performance-related parameters include timing data; analyzing, using the one or more processors, the subject's performance as a combination of performance-related parameters selected across the one or more tasks presented to the subject; and generating, using the one or more processors, a cognitive measurement of the subject based on the analyzed performance.

In another embodiment, the one or more tasks presented to the subject comprise at least one of: a temporal numerosity judgment task, a rhythm discrimination task, a pitch discrimination task, and a simple reaction-time task.

In yet another embodiment, the temporal numerosity judgment task comprises presenting a variable number of visual flashes and auditory tones; recording the subject's judgment of the number of flashes and tones; and analyzing an accuracy and precision of the subject's judgments.

In a further embodiment, the rhythm discrimination task comprises presenting sequences of visual or auditory stimuli with varying time intervals; recording the subject's responses in comparing, matching, or judging the rhythms; and assessing the subject's accuracy, precision, and/or thresholds in perceiving the rhythms.

In an additional embodiment, the pitch discrimination task comprises presenting a sequence of auditory tones to the subject, wherein each tone has a specific frequency; instructing the subject to identify whether consecutive tones are the same or different in pitch; recording the subject's responses and response times for each pair of tones; adjusting the frequency difference between tones based on the subject's performance to determine a pitch discrimination threshold; and analyzing the subject's pitch discrimination ability based on the recorded responses, response times, and determined threshold.

In another embodiment, generating the cognitive measurement comprises generating a global score based on the subject's performance across the one or more tasks.

In yet another embodiment, the one or more tasks utilizes only one sensory modality of either visual and auditory.

In a further embodiment, the one or more tasks utilizes a combination of both visual and auditory modality.

In an additional embodiment, the method further comprises determining a pattern of performance based on the subject's performance across the one or more tasks.

In another embodiment, the global score is generated by normalizing scores from different types of tasks and calculating a weighted average of the normalized scores.

In one embodiment, a method for cognitive training is provided. The method comprises determining, using a training device, a training level based on a cognitive measurement of a subject; presenting, using the training device, one or more tasks to the subject, wherein the one or more tasks include a time series of visual stimuli, audio stimuli, or both visual and audio stimuli for cognitive training based on the determined training level; collecting, using the training device, responses from the subject in response to the presented stimuli; calculating, using one or more processors, timing and performance related parameters based on the collected responses; analyzing, using the one or more processors, the subject's performance based on the calculated timing and performance related parameters; determining, using the one or more processors, the subject's performance relative to the cognitive training; and adjusting, using the one or more processors, the training level based on the determined performance.

In another embodiment, adjusting the training level comprises increasing or decreasing a difficulty of the visual task, auditory task, or both sensorimotor task presented to the subject.

In yet another embodiment, the cognitive training comprises at least one of: a computerized task, and an immersive task.

In a further embodiment, the computerized task comprises presenting concurrent visual and auditory stimuli to the subject, wherein the stimuli consist of sequences of brief visual and auditory events presented in rapid succession; instructing the subject to process a temporal structure of the visual and auditory stimuli; prompting the subject to perform at least one selected from the group consisting of: determining whether two audiovisual sequences are the same or different; determining whether the auditory and visual sequences match; responding with temporal precision to the end of a sequence in one modality cued by another modality; and providing feedback to the subject on each trial about a correctness and speed of their response.

In an additional embodiment, the immersive task comprises presenting concurrent rhythmic visual, auditory, and tactile stimuli to the subject in a simulated environment; instructing the subject to process and respond to the concurrent stimuli with temporal precision; prompting the subject to perform actions in response to the concurrent stimuli, wherein the actions simulate interaction with virtual objects; tracking the subject's movements and responses in relation to the presented stimuli; providing real-time feedback to the subject on their performance; and adaptively adjusting a difficulty of the task based on the subject's performance.

In another embodiment, the immersive task is delivered through a virtual reality interface.

In yet another embodiment, the virtual reality interface presents gamified cognitive training exercises that combine cognitive tasks with physical activities, and utilize a musical rhythmic game.

In a further embodiment, the method further comprises presenting a 40 Hz flickering visual stimulus and a 40 Hz amplitude-modulated sound during breaks in the cognitive training.

In an additional embodiment, calculating timing and performance-related parameters comprises recording accuracy, reaction times, and consistency across multiple training sessions.

In one embodiment, a cognitive assessment and training system is provided. The system comprises a memory; one or more processors; and an input/output device; wherein the one or more processors are configured to execute instructions stored in the memory to: present one or more assessment tasks to a subject via the input/output device, wherein the one or more tasks include a time series of visual stimuli, audio stimuli, or both visual and audio stimuli, wherein the visual stimuli comprises flashes of light and the audio stimuli comprises short auditory tones, wherein the short audio tones comprises noise bursts; receive response data from the subject for each of the one or more tasks via the input/output device; calculate performance-related parameters based on the response data, wherein the performance-related parameters include timing data; analyze the subject's performance as a combination of performance-related parameters selected across the one or more tasks presented to the subject; generate a cognitive measurement of the subject based on the analyzed performance; determine a training level based on the cognitive measurement; present a time series of visual stimuli, audio stimuli, or both visual and audio stimuli to the subject for cognitive training via the input/output device based on the determined training level; collect responses from the subject in response to the presented stimuli; calculate timing and performance related parameters based on the collected responses; analyze the subject's performance based on the calculated timing and performance related parameters; determine the subject's performance relative to the cognitive training; and adjust the training level based on the determined performance.

In another embodiment, the one or more assessment tasks comprise at least one of: a temporal numerosity judgment task, a rhythm discrimination task, a pitch discrimination task, and a simple reaction-time task.

In yet another embodiment, the temporal numerosity judgment task comprises presenting a variable number of visual flashes and auditory tones; recording the subject's judgment of the number of flashes and tones; and analyzing an accuracy and precision of the subject's judgments.

In a further embodiment, the rhythm discrimination task comprises presenting sequences of visual or auditory stimuli with varying time intervals; recording the subject's responses in comparing, matching, or judging the rhythms; and assessing the subject's accuracy, precision, and/or thresholds in perceiving the rhythms.

In an additional embodiment, the pitch discrimination task comprises presenting a sequence of auditory tones to the subject, wherein each tone has a specific frequency; instructing the subject to identify whether consecutive tones are the same or different in pitch; recording the subject's responses and response times for each pair of tones; adjusting the frequency difference between tones based on the subject's performance to determine a pitch discrimination threshold; and analyzing the subject's pitch discrimination ability based on the recorded responses, response times, and determined threshold.

In another embodiment, the cognitive training comprises at least one of: a computerized task, and an immersive task.

In yet another embodiment, the computerized task comprises presenting concurrent visual and auditory stimuli to the subject, wherein the stimuli consist of sequences of brief visual and auditory events presented in rapid succession; instructing the subject to process a temporal structure of the visual and auditory stimuli; prompting the subject to perform at least one selected from the group consisting of: determining whether two audiovisual sequences are the same or different; determining whether the auditory and visual sequences match; responding with temporal precision to the end of a sequence in one modality cued by another modality; and providing feedback to the subject on each trial about a correctness and speed of their response.

In a further embodiment, the immersive task comprises presenting concurrent rhythmic visual, auditory, and tactile stimuli to the subject in a simulated environment; instructing the subject to process and respond to the concurrent stimuli with temporal precision; prompting the subject to perform actions in response to the concurrent stimuli, wherein the actions simulate interaction with virtual objects; tracking the subject's movements and responses in relation to the presented stimuli; providing real-time feedback to the subject on their performance; and adaptively adjusting a difficulty of the task based on the subject's performance.

In an additional embodiment, generating the cognitive measurement comprises generating a global score based on the subject's performance across the one or more tasks.

In another embodiment, the global score is generated by normalizing scores from different types of tasks and calculating a weighted average of the normalized scores.

In yet another embodiment, the one or more processors are further configured to present a 40 Hz light flickering visual stimulus and a 40 Hz amplitude-modulated sound during breaks in the cognitive training.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a cognitive assessment and rehabilitation system in accordance with an embodiment of the invention.

FIG. 2 illustrates a cognitive assessment process in accordance with an embodiment of the invention.

FIG. 3 illustrates a cognitive training process in accordance with an embodiment of the invention.

FIG. 4 illustrates correlations between specific perceptual abilities and cognitive abilities in accordance with an embodiment of the invention.

FIG. 5 illustrates a cognitive assessment element in accordance with an embodiment of the invention.

FIG. 6 illustrates a cognitive assessment application in accordance with an embodiment of the invention.

FIG. 7 illustrates a cognitive training element in accordance with an embodiment of the invention.

FIG. 8 illustrates a cognitive training application in accordance with an embodiment of the invention.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

In many embodiments of the invention, cognitive assessment and rehabilitation systems provide innovative approaches to assessing and improving cognitive function. These systems utilize novel computerized behavioral tasks for cognitive assessment, including temporal numerosity judgment tasks, rhythm discrimination tasks, simple reaction time tasks, and auditory-visual pitch discrimination tasks. The performance on these tasks has been discovered to correlate with and be predictive of higher-level cognitive abilities, a relationship that was previously unknown.

Cognitive assessment and rehabilitation systems may generate a global assessment score based on performance across multiple tasks. This score often involves using a weighted average of normalized scores from individual tasks. In several embodiments, these scores can be mapped onto established cognitive tests, such as Raven's progressive matrices, digit span, and verbal memory tests. This mapping may be useful for assessing intelligence, memory, concussion, Alzheimer's Disease, and cognitive decline in aging.

In a number of embodiments, cognitive assessment and rehabilitation systems incorporate multiple training protocols and techniques for cognitive rehabilitation. These may include computerized training on desktop computers, laptops, or tablets that provide passive exposure to visual and auditory stimuli and utilize multisensory perceptual learning paradigms. Some embodiments may also include Virtual Reality (VR) training, which often involves gamified tasks and motor behavior in response to dynamic auditory-visual stimuli.

Cognitive assessment and rehabilitation systems may aim to improve both unisensory processing (auditory and visual) and multisensory integration. In many embodiments, these systems offer potential benefits including improvement in memory and cognition, as well as therapeutic benefits for individuals experiencing cognitive decline and dementia. Some implementations may feature adaptive difficulty levels that adjust based on user performance, allowing for personalized training experiences.

It is important to note that the characteristics described here do not reflect all embodiments of the invention, as cognitive assessment and rehabilitation systems may vary in their specific implementations and features. The use of these various tasks for cognitive assessment and rehabilitation represents a novel approach in the field, leveraging the newly discovered relationship between performance on these tasks and broader cognitive abilities.

In many embodiments of the invention, cognitive assessment and rehabilitation systems provide comprehensive platforms for evaluating and improving cognitive abilities. These systems employ modular architectures that may be implemented on individual user devices or distributed across servers accessible through various user interfaces. Cognitive assessment and rehabilitation systems support specific functions including assessment delivery, data analysis, personalized training regimens, and progress tracking, working in concert to provide holistic approaches to cognitive enhancement.

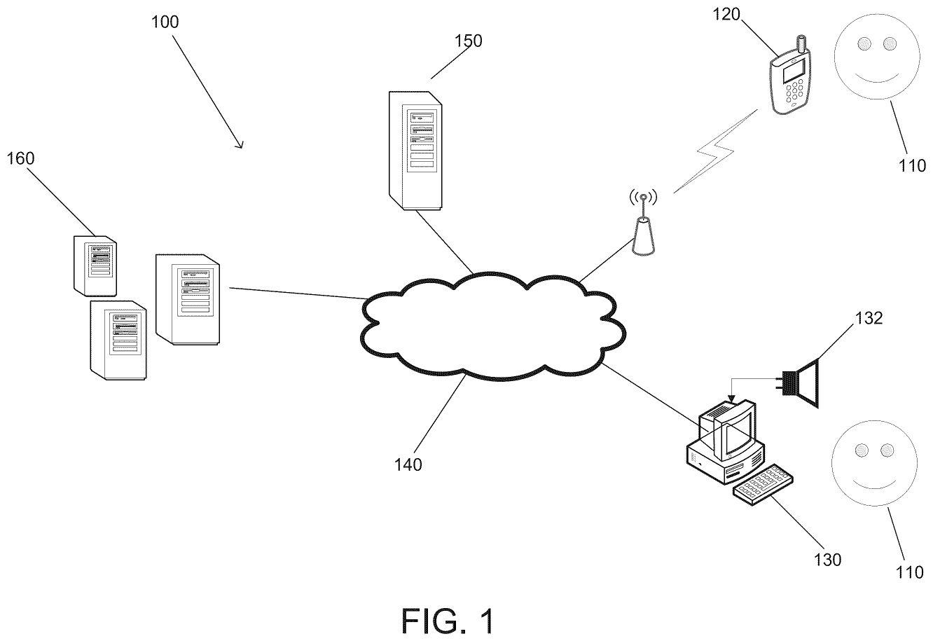

A cognitive assessment and rehabilitation system in accordance with an embodiment of the invention is illustrated in FIG. 1. The cognitive assessment and rehabilitation system 100 comprises several interconnected components. A user 110 interacts with the cognitive assessment and rehabilitation system 100 through either a mobile device 120 or a computing device 130, which serves as a primary interface for assessment tasks and rehabilitation exercises. The mobile device 120 may be equipped with a touchscreen display and various sensors to capture user inputs and responses.

The mobile device 120 and/or the computing device 130 can process data collected during assessment and rehabilitation sessions. The computing device 130 may be a standalone unit or integrated within the mobile device 120, depending on the specific implementation of the cognitive assessment and rehabilitation system 100.

When the user 110 accesses the cognitive assessment and rehabilitation system 100 through the computing device 130, a speaker 132 is included as a separate component of the cognitive assessment and rehabilitation system 100. The speaker 132 outputs audio content during assessment and rehabilitation sessions, providing auditory stimuli and instructions to the user 110 as required by various cognitive tasks.

The cognitive assessment and rehabilitation system 100 leverages cloud infrastructure 140 to store and process large amounts of data. The cloud 140 is connected to a server 150, which manages the distribution of tasks, data analysis, and the generation of personalized cognitive training programs based on the user's performance.

For more extensive processing capabilities, the cognitive assessment and rehabilitation system 100 may utilize a group of servers 160. The group of servers 160 work in tandem to handle complex computations, machine learning algorithms, and data mining operations that contribute to the cognitive assessment and rehabilitation system's adaptive and personalized approach to cognitive assessment and rehabilitation.

Various processes for cognitive assessment and rehabilitation are discussed above with reference to FIG. 1. Alternative processes may be utilized as appropriate to the requirements of specific applications. These alternative processes also perform cognitive assessment and rehabilitation. These alternatives may provide accurate and robust assessment and rehabilitation of cognitive abilities in accordance with various embodiments of the invention.

Cognitive assessment processes provide valuable insights into an individual's mental capabilities and cognitive function. These assessments may be utilized in rehabilitation programs to improve cognitive abilities across various populations. The process typically involves a comprehensive evaluation using multiple tasks, which collectively contribute to a global score representing an overall measure of cognitive ability. This multifaceted approach allows for a thorough assessment of different cognitive domains, providing a holistic view of an individual's cognitive strengths and areas for potential improvement.

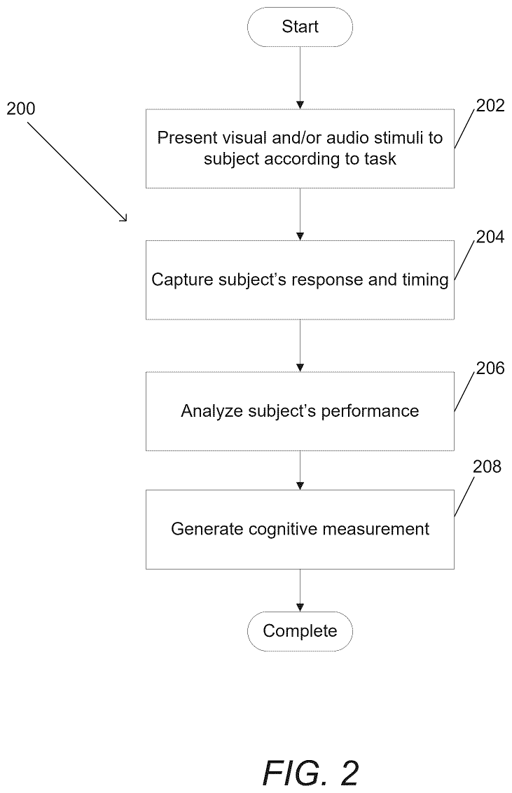

A cognitive assessment process in accordance with an embodiment of the invention is illustrated in FIG. 2. The cognitive assessment process begins with presenting visual and/or audio stimuli to a subject according to a task 202. The task may be selected from a variety of options, including but is not limited to a temporal numerosity judgment task (counting number of flashes and beeps, as in Shams, L., Kamitani, Y., & Shimojo, S., What you see is what you hear, Nature, Vol. 408, pp. 788 (2000); Odegaard, B., Shams, L., Brain's tendency to combine auditory and visual signals is stable but not general, Psychological Science, doi: 10.1177/0956797616628860 (2016)), a rhythm discrimination task (as in Barakat, B. K., Seitz, A. R., Shams, L., Visual rhythm perception improves through auditory, but not visual, training, Current Biology. Vol 25(2), pp. 60-61 (2015)), an auditory-visual simple reaction time task or pitch discrimination task. In some cases, these tasks may be gamified to enhance engagement and motivation.

The cognitive assessment process captures the subject's response and timing 204. Processes in accordance with many embodiments records the accuracy of the subject's responses as well as the time taken to respond to each stimulus. The data collected during this phase forms the basis for subsequent analysis.

Following data collection, the cognitive assessment process proceeds to analyze the subject's performance 206. This analysis may employ various methods, including traditional analytical approaches, a Bayesian computation model, or a diffusion model. Each of these methods offers unique insights into the subject's cognitive processes and abilities. Bayesian computation models in accordance with selected embodiments may include the Bayesian Causal Inference model discussed in Odegaard & Shams, Psychological Science 2016 (cited above) or Zhu, H., Beierholm, U., Shams, L. (2024) BCI Toolbox: an open-source python package for Bayesian Causal Inference. PLoS Computational Biology. https://doi.org/10.1371/journal.pcbi.1011791. In certain embodiments, diffusion models, such as the Hierarchical Drift Diffusion Model as discussed in Wiecki T, Sofer I, Frank M., HDDM: hierarchical bayesian estimation of the drift-diffusion model in python, Frontiers in Neuroinformatics 7:14 (2013) may be used for detection, characterization, and quantification of multisensory integration in behavioral tasks in which both accuracy and reaction time data are available. Various other models such as those based on Signal Detection Theory (e.g., Rosenthal, O. Shimojo, S., Shams, L., Sound-induced flash illusion is resistant to feedback training, Brain Topography, Vol 21, pp. 185-192 (2009)) as appropriate to the requirements of specific application may also be utilized to provide a rigorous measure of an individual's tendency/capacity to integrate auditory and visual signals, as well as unisensory precision/sensitivity.

Process 200 generates 208 a cognitive measurement. This measurement may be a global score derived from the subject's performance across multiple tasks. In some cases, the global score may be obtained through the use of models and weighted averages of individual task scores.

It is important to note that a user may only need to interact with one, two, or all of the tests in either audio or visual modality, as the assessment may only require unisensory assessment as input. This flexibility allows for tailored assessments based on specific cognitive domains of interest or individual capabilities.

The performance on these tasks may be used to predict cognitive abilities. The scores obtained from these assessments can be mapped onto established cognitive tests commonly used to measure intelligence, memory, and other cognitive functions. This mapping allows for predictions of performance on tests such as Raven's matrices, digit span, verbal memory, trail task, and cancellation task. These predictions may be valuable in assessing intelligence, memory, concussion, Alzheimer's Disease, and cognitive decline in aging.

Various processes for cognitive assessment are discussed above with reference to FIG. 2. Alternative processes may be utilized as appropriate to the requirements of specific applications. These alternative processes also perform cognitive assessment. These alternatives may provide accurate and robust assessment of cognitive abilities in accordance with various embodiments of the invention.

The temporal numerosity judgment task involves presenting a variable number of flashes and beeps simultaneously. The number of flashes and beeps may vary from zero to three or four. The flashes may be small visual stimuli presented for a few milliseconds each, while the beeps may be tones or clicks presented for a few milliseconds each. The time interval between consecutive flashes or beeps may be brief (e.g., 60 ms), selected such that the task of counting flashes and beeps is moderately challenging but not impossible for a healthy young individual. The subject's task is to judge the number of flashes and beeps on each trial.

In some embodiments of the invention, there will be a subset of trials in which only beeps are presented (auditory trials) and/or a subset of trials in which only flashes are presented (visual trials). Multiple repetitions of each trial type will be presented. Furthermore, all the visual, auditory, and auditory-visual trials can be presented in a pseudo-random order (interleaved).

The task is for the subject to count the number of flashes and/or the number of beeps on each trial. In some embodiments, the task can be presented as unisensory blocks, in which only beeps or only flashes are presented and the task is to report only the number of beeps or only the number of flashes, respectively. The responses to all trials are recorded (e.g., the number of flashes and/or beeps counted by the subject vs. the number that was presented) and the response time may also be recorded.

The subject's performance (accuracy, reaction time) can be quantified in the following sets of trials: unisensory auditory trials within the bisensory block, unisensory visual trials within the bisensory block, unisensory auditory trials within the auditory block, unisensory visual trials within the visual block, auditory bias of visual perception in the bisensory trials. Bayesian Causal Inference modeling may also be applied to the data to estimate perceptual processing parameters. Pcommon parameter (prior probability of integration), sigma_A (auditory noise), and sigma_V (visual noise) can be estimated using the model. Signal detection theory analysis may be used to assess changes in sensitivity caused by interaction between the two senses as a measure of audiovisual (AV) integration.

In addition, the sum of auditory accuracy and visual accuracy will be computed as a measure of unisensory acuity. The reciprocal of the sum of auditory and visual noise parameters (1/(sigma_A+sigma_V)) can also be computed as a measure of unisensory acuity.

The rhythm discrimination task may involve presenting sequences of visual or auditory stimuli with varying time intervals between them. For visual sequences, multiple flashes with varying time intervals may be presented. For auditory sequences, multiple beeps with varying time intervals may be presented. The subject's accuracy, precision, and/or thresholds in perceiving the rhythms may be assessed through tasks such as comparing different sequences of rhythms, matching rhythms, or making judgments about the rhythms.

The sequence includes multiple flashes or multiple beeps with varying time intervals between them. The time intervals will be brief (e.g., ranging from 50-200 ms).

In the auditory block, two sequences of beeps (e.g., tones or noisebursts) will be presented in two intervals and the task will be to judge whether the two sequences were the same or different. Each beep will be brief (a few milliseconds), the sequence will consist of multiple beeps (e.g., 6 or 7) and the time interval between the beeps will define the sequence/rhythm, and will vary from trial to trial or between intervals. In the visual block, two sequences of flashes (e.g., small white disk in the center of the visual field on a black bakcground) will be presented in two intervals and the task will be to judge whether the two sequences were the same or different. Each flash will be brief (a few milliseconds), the sequence will consist of multiple flashes (e.g., 6 or 7) and the time interval between the flashes will define the sequence/rhythm, and will vary from trial to trial or between intervals. There may be a third block of trials in which both flashes and beeps will be presented in each interval. The flashes and beeps will be consistent both in duration and timing, and the task will be to judge whether the two AV sequences were the same or different.

The auditory-visual motion discrimination task may involve presenting random dot kinematograms, which consist of a set of dots moving with a certain subset of the dots moving in the same direction. The task difficulty may be adjusted by varying the proportion of dots moving in the same direction. The subject's accuracy and precision in perceiving the motions may be assessed through tasks such as detecting motion, comparing different motions, matching motions, or making judgments about the motions. In the detection task, one interval will contain coherent motion, and one interval contains no coherent motion, and the task will be to judge which interval contained coherent motion. In the discrimination task, both intervals contain coherent motion, but the direction of motion may be the same or different, and the task is to judge whether the two directions were the same or different.

For the various other tasks some of which are listed above, unisensory accuracy (measured from the unisensory trials) can be computed. Additionally, signal detection analysis methods can be used to quantify the sensitivity in detection or discrimination in unisensory and multisensory conditions.

If more than one task is completed, the overall unisensory acuity score can be computed by taking the normalized average of the unisensory accuracy/noise/reaction-time/sensitivity measured from the different tasks. Similarly, an overall multisensory integration score can be computed by taking the average of the normalized average of multisensory integration scores across the different tasks.

Cognitive training processes play a vital role in improving cognitive function and rehabilitating individuals experiencing cognitive decline. These processes leverage cognitive measurements obtained through assessment tasks to develop personalized training regimens. Training may manifest in various formats, including computerized tasks, virtual reality games, and neurofeedback sessions. The cognitive training approach aims to enhance both unisensory processing and multisensory integration, potentially leading to improvements in memory, cognition, and overall cognitive abilities.

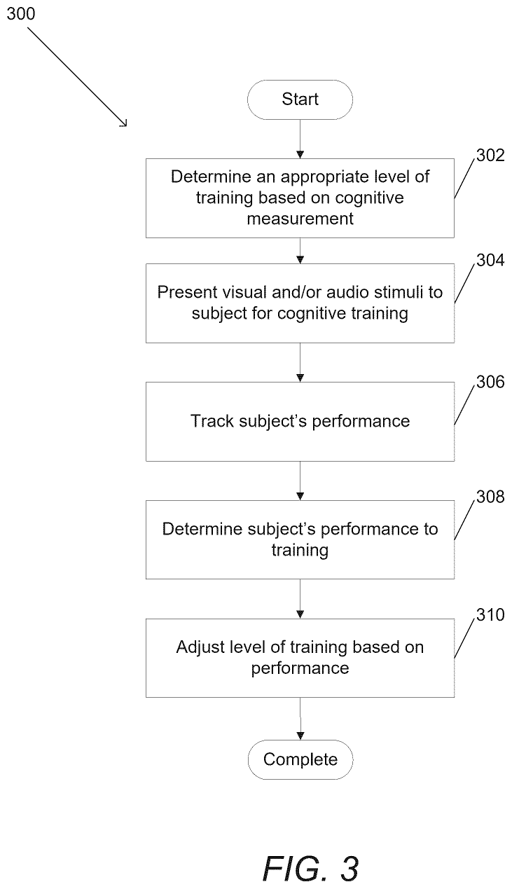

A cognitive training process in accordance with an embodiment of the invention is illustrated in FIG. 3. The cognitive training process begins with determining an appropriate level of training based on cognitive measurement 302. Processes in accordance with various embodiments utilize the cognitive measurements obtained from previous assessment tasks to tailor the training difficulty to the individual's current cognitive abilities.

The cognitive training process continues with presenting visual and/or audio stimuli to the subject for cognitive training 304. These stimuli may take various forms depending on the specific training protocol being employed. For computerized training, tasks may involve exposure to visual stimuli such as flashes and auditory stimuli such as noise bursts. These tasks often require processing of temporal structures and responding to audiovisual sequences.

In some cases, the training may utilize virtual reality technology. The virtual reality training may present a gamified environment where the subject interacts with dynamic audiovisual stimuli. The subject may be required to perform actions in response to rhythmic visual and auditory cues, similar to popular rhythm-based games. For example, the subject may need to hit or slice virtual objects in sync with music in a three-dimensional virtual space. This approach combines cognitive training with physical activity, potentially enhancing engagement and effectiveness.

As the cognitive training process progresses, the system tracks the subject's performance 306. This tracking may involve recording accuracy, reaction times, and other relevant metrics specific to the training tasks being performed.

Following the tracking, the cognitive training process involves determining the subject's performance to training 308. Processes in accordance with several embodiments analyze the collected performance data to assess how well the subject is responding to the current level of training.

Based on this analysis, the cognitive training process may adjust the level of training based on performance 310. If the subject is performing well, the difficulty may be increased to provide continued challenge and improvement. Conversely, if the subject is struggling, the difficulty may be decreased to ensure the training remains engaging and beneficial.

In some implementations, the training may incorporate specific audiovisual manipulations known to be beneficial for brain health. For instance, visual stimuli may be presented with a 40 Hz flicker, while auditory stimuli may be convolved with a 40 Hz frequency. These manipulations may help entrain certain brain wave patterns associated with improved cognitive function.

Various processes for cognitive training and rehabilitation are discussed above with reference to FIG. 3. Alternative processes may be utilized as appropriate to the requirements of specific applications. These alternative processes also perform cognitive training and rehabilitation. These alternatives may provide accurate and robust rehabilitation of cognitive abilities in accordance with various embodiments of the invention.

Computerized training tasks in accordance with a number of embodiments of the invention can involve concurrent visual and auditory stimuli. The stimuli can consist of sequences of brief visual and auditory stimuli presented in rapid succession. The task would require processing of the temporal structure of the visual and auditory stimuli and responding whether two audiovisual (AV) sequences are the same or different, or whether the A and V sequences match, or responding with temporal precision to the end of the sequence in one modality cued by another modality, or another similar task requiring the precise and quick processing of temporal information across the two modalities. This kind of training is akin to musical training, but without the use of a musical instrument, and with an emphasis on the temporal and rhythmic aspects of audiovisual stimuli. The user receives feedback on each trial about the correctness (and may also include the speed) of their response. In several embodiments, the user performs one or a combination of these tasks multiple times a week, each time for at least 10 minutes. These simple tasks can be gamified, and user may receive “rewards” (e.g., in the form of animation or sound effects) as they improve.

Training in virtual reality in accordance with embodiments of the invention can allow for different levels of mobility and physical exertion. For example, at the lowest level, the player can play sitting down and only moving arms. At the highest level, the player moves around frequently and expends energy. The task involves processing rhythmic visuals and sounds (and may also include tactile stimuli), and responding with temporal precision. The games may be similar to games for learning a musical instrument, such as drums, xylophone, bottles, etc. involving both visual and auditory sensory inputs, and ideally tactile as well.

Depending on the physical fitness level selected from an initial menu, there can be short intervals between the musical performance tasks with physical activities (e.g., squatting, bending). As the performance improves, the player should process more challenging AV time sequences, and has to respond with more temporal precision. The player can receive tactile feedback, in addition to visual and auditory feedback. The player can choose the setting, and in some embodiments even the music (from a selection). Customization can allow for more enjoyment and more engagement. The visual size and contrast can be adjustable to accommodate older adults with low vision. The sound loudness can be adjustable to accommodate older adults with hearing impairment. The game difficulty can be adaptive to the player's performance, so that it does not remain too difficult or too easy for too long. Games can be relatively short (e.g., 5-7 minutes), to allow breaks to avoid motion sickness.

Each time the player ‘scores’ or intermittently after improvements, the player can receive a positive sensory feedback. Player may have an option to take a “break”. The break period may be a few minutes and can be stopped at any time. During “break”, a 40 Hz light flicker (in the peripheral vision) and 40 Hz sound can be presented while the player views pleasant stimuli (e.g., nature images) at the center of the screen. This can help entrain Gamma oscillations in the brain which have been shown to be beneficial for improving cognition (sec., e.g., He Q, Colon-Motas K, Pybus A, Piendel L, Seppa J, Walker M, Manzanares C, Qui D, Miocinovic S, Wood L, Levey A, Lah J, Singer AC, “A feasibility trial of gamma sensory Flicker for patients with prodromal Alzheimer's Disease.” Alzheimer's & Dementia: TRCI https://doi.org/10.1002/trc2.12178 (2021); Martorell AJ*, A L Paulson*, H-J Suk, F Abdurrob, GT Drummond, W Guan, JZ Young, DN-W Kim, O Kritskiy, SJ Baker, V Mangena, SM Prince, EN Brown, KC Chung, E S Boyden, AC Singer, L-H Tsai, Multi-sensory gamma stimulation ameliorates Alzheimer's-associated pathology and improves cognition, Cell, https://www.cell.com/cell/fulltext/S0092-8674(19)30163-1 (2019)).

In addition to the computerized and virtual reality training methods described above, some embodiments may employ neurofeedback training as a rehabilitation tool. In neurofeedback training, a small set of electroencephalography (EEG) electrodes may record electrical activity in the brain. Brain activity may be analyzed in real-time and present visual and/or auditory stimuli accordingly. This feedback may serve to gradually train the brain for improved unisensory and multisensory processing.

The neurofeedback training may be customized to individual users. Brain activity in response to auditory, visual, and auditory-visual stimuli may be measured and used to train a machine learning decoder or classifier. This classifier may extract signatures of successful auditory or visual processing, or auditory-visual integration in the individual's brain. These signatures may then be used to guide the neurofeedback training, aiming to enhance the brain's ability to process unisensory information or integrate multisensory inputs.

These various training approaches—computerized tasks, virtual reality games, and neurofeedback sessions—may be employed individually or in combination, depending on the specific needs and goals of the cognitive rehabilitation program. By targeting both unisensory processing and multisensory integration, these training methods aim to produce cognitive improvements that may generalize to real-life tasks and functions, potentially offering therapeutic benefits for individuals experiencing cognitive decline or dementia.

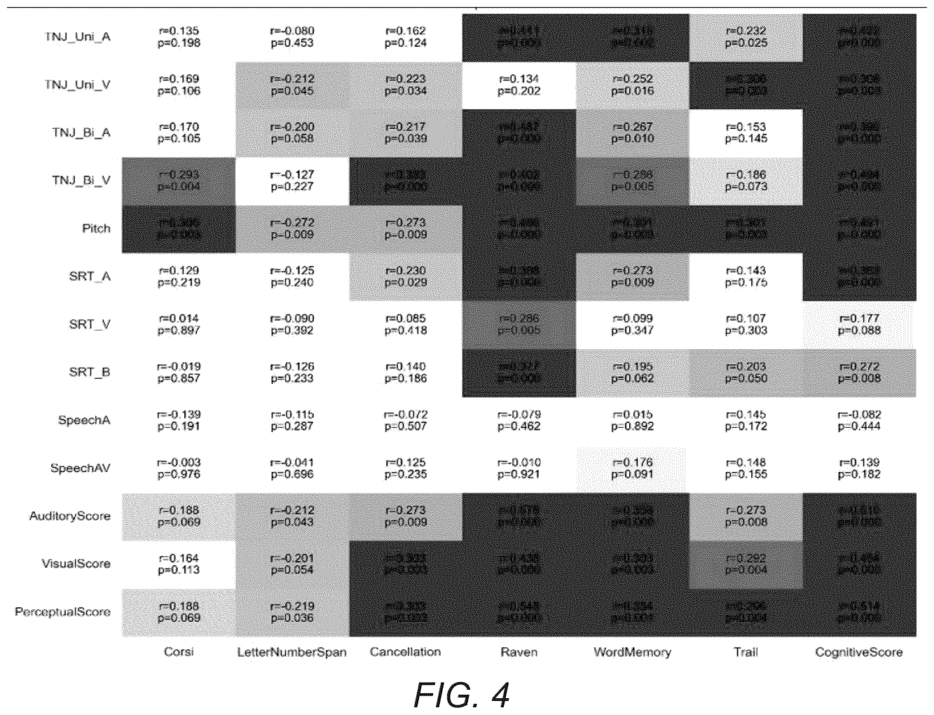

FIG. 4 illustrates correlations between specific perceptual abilities and cognitive abilities in accordance with an embodiment of the invention. The correlation coefficient r, and statistical significance, p value, is shown for data obtained from 98 young adult participants who performed a set of perceptual tasks (shown across rows) and a battery of cognitive tests (shown across columns). For illustration purposes, only a subset of perceptual tasks are represented. The color of each box represents the statistical significance of the correlation, with dark red denoting highest significance level, and white denoting lowest p value (no statistical significance). The final column “cognitive score” is the sum of normalized scores across all cognitive tests. The “auditory score” and “visual score” are the sum of normalized auditory task scores, and sum of normalized visual task scores, respectively, for tasks that were strongly correlated with cognition (namely, TNJ, pitch, SRT). “Perceptual score” represents the sum of visual and auditory scores. TNJ_A and TNJ_V denote the tasks of counting beeps and flashes, respectively. “Uni” and “bi” represent the unisensory vs. bisensory blocks of trials. SRT_A and SRT_V and SRT_B denote the simple reaction time task to visual, auditory, or auditory-visual stimuli, respectively. “Pitch” represents the pitch discrimination task. speech_A and speech_AV represent a word in noise identification task in auditory or auditory-visual conditions, respectively. Not all perceptual tasks showed a correlation with cognitive scores, as can be seen in speech tasks. The results shown here are the second replication of findings on some of the tasks (e.g., TNJ). Previous two studies had similarly large sample sizes of ˜100.

Cognitive assessment elements provide modular architectures for evaluating cognitive abilities through computerized behavioral tasks. These elements may be implemented as standalone computing devices or integrated within mobile devices, offering flexibility in deployment and user interaction. Cognitive assessment elements typically incorporate processing capabilities, data storage, network connectivity, and specialized software applications to deliver assessment tasks, analyze performance data, and generate cognitive measurements.

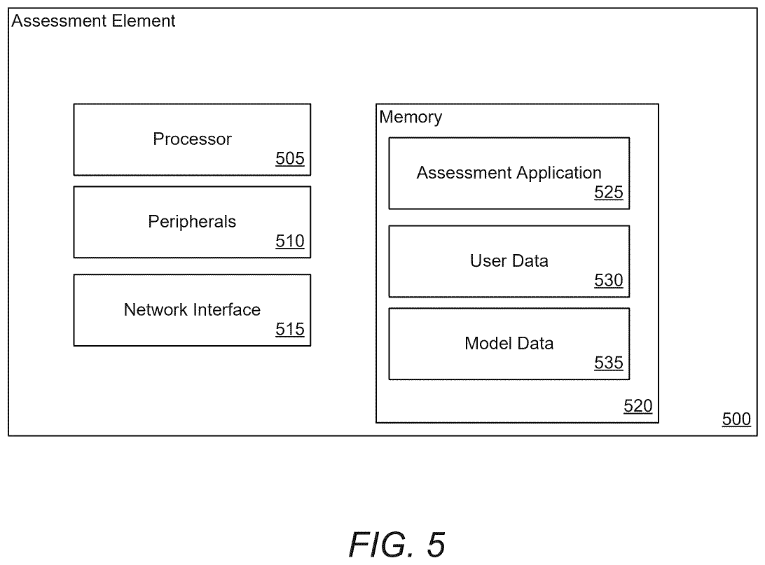

A cognitive assessment element in accordance with an embodiment of the invention is illustrated in FIG. 5. The assessment element 500 comprises several interconnected components that work together to deliver cognitive assessment functionality.

The assessment element 500 includes a processor 505 that serves as the central computing unit for the element. The processor 505 may execute instructions stored in memory and perform calculations necessary for delivering assessment tasks, analyzing user responses, and generating cognitive measurements. In some cases, the processor 505 may be a multi-core processor capable of parallel processing to enhance performance.

The assessment element 500 also incorporates peripherals 510, which may include input/output devices such as displays, touchscreens, keyboards, and audio systems. These peripherals 510 enable user interaction with the assessment element 500, allowing for the presentation of visual and auditory stimuli and the capture of user responses during assessment tasks.

A network interface 515 is included in the assessment element 500 to facilitate communication with external systems and services. The network interface 515 may support various connectivity options such as Wi-Fi, cellular data, or Ethernet, enabling the assessment element 500 to transmit and receive data, access cloud-based resources, and integrate with larger cognitive assessment and rehabilitation systems.

The assessment element 500 contains memory 520, which may include both volatile and non-volatile storage components. The memory 520 stores the operating system, application software, and data necessary for the functioning of the assessment element 500. In some cases, the memory 520 may also cache assessment tasks and user data to improve performance and reduce network dependency.

An assessment application 525 resides within the memory 520 of the assessment element 500. The assessment application 525 may contain the logic and processes necessary to deliver cognitive assessment tasks, process user responses, and generate cognitive measurements. In some cases, the assessment application 525 may be modular, allowing for easy updates and customization of assessment protocols.

The assessment element 500 maintains user data 530 within its memory. The user data 530 may include historical performance records, demographic information, and other relevant data specific to individual users. This data may be used to personalize assessment tasks and track cognitive changes over time.

Model data 535 is also stored within the assessment element 500. The model data 535 may contain parameters and weights for computational models used in analyzing assessment performance and generating cognitive measurements. In some cases, the model data 535 may be updated regularly to incorporate the latest research findings and improve assessment accuracy.

Various elements for a cognitive assessment element are discussed above with reference to FIG. 5. Alternative elements may be utilized as appropriate to the requirements of specific applications. These alternative elements also perform cognitive assessment. These alternatives may provide accurate and robust assessment of cognitive abilities in accordance with various embodiments of the invention.

Cognitive assessment applications provide modular software architectures for evaluating cognitive abilities through computerized behavioral tasks. These applications may be implemented on various computing devices, including personal computers, tablets, and smartphones, offering flexibility in deployment and user interaction. Cognitive assessment applications typically incorporate specialized modules for delivering assessment tasks, analyzing performance data, and generating cognitive measurements.

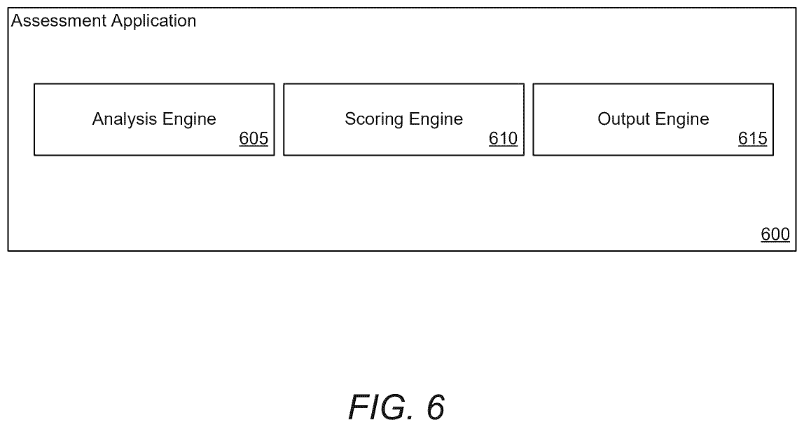

A cognitive assessment application in accordance with an embodiment of the invention is illustrated in FIG. 6. The assessment application 600 includes an analysis engine 605 that serves as the core processing unit for cognitive assessments. The analysis engine 605 may execute processes to process user responses from assessment tasks, perform statistical analyses, and generate preliminary cognitive measurements. In some cases, the analysis engine 605 may utilize machine learning models to enhance the accuracy of assessments.

A scoring engine 610 is incorporated within the assessment application 600. The scoring engine 610 may take the preliminary measurements from the analysis engine 605 and apply standardized scoring protocols to generate final cognitive scores. The scoring engine 610 may also normalize scores across different assessment tasks and compare results to population norms stored in the model data.

The assessment application 600 contains an output engine 615 to present assessment results and cognitive measurements. The output engine 615 may generate visual representations of cognitive scores, create detailed reports, and prepare data for transmission to external systems or healthcare providers. In some cases, the output engine 615 may also provide personalized feedback and recommendations based on assessment results.

The analysis engine 605, scoring engine 610, and output engine 615 work in concert to deliver comprehensive cognitive assessments. The analysis engine 605 may process raw data from assessment tasks and pass preliminary results to the scoring engine 610. The scoring engine 610 may then apply standardized protocols and normalization techniques before sending final scores to the output engine 615 for presentation and reporting.

Various applications for cognitive assessment are discussed above with reference to FIG. 6. Alternative applications may be utilized as appropriate to the requirements of specific applications. These alternative applications also perform cognitive assessment. These alternatives may provide accurate and robust assessment of cognitive abilities in accordance with various embodiments of the invention.

Cognitive training elements provide modular architectures for delivering personalized cognitive rehabilitation exercises and tracking progress over time. These elements may be implemented as standalone computing devices or integrated within mobile devices, offering flexibility in deployment and user interaction. Cognitive training elements typically incorporate processing capabilities, data storage, network connectivity, and specialized software applications to deliver training tasks, analyze performance data, and adapt difficulty levels based on user progress. In some cases, cognitive training elements may be implemented using the same hardware as cognitive assessment elements, allowing for seamless integration of assessment and rehabilitation functions.

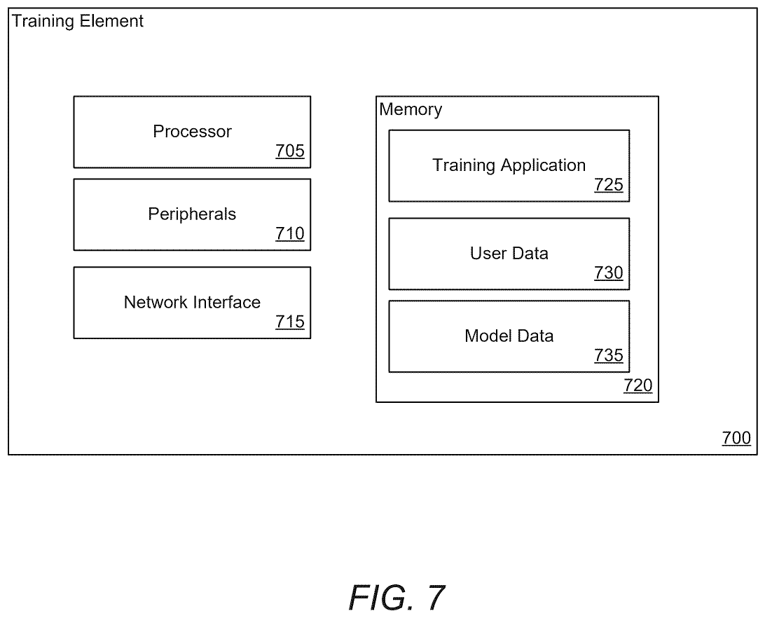

A cognitive training element in accordance with an embodiment of the invention is illustrated in FIG. 7. The training element 700 comprises several interconnected components that work together to deliver cognitive training functionality.

The training element 700 includes a processor 705 that serves as the central computing unit for the element. The processor 705 may execute instructions stored in memory and perform calculations necessary for delivering training tasks, analyzing user performance, and adapting difficulty levels. In some cases, the processor 705 may be a multi-core processor capable of parallel processing to enhance performance.

The training element 700 also incorporates peripherals 710, which may include input/output devices such as displays, touchscreens, keyboards, and audio systems. In some cases, the peripherals 710 may include VR headsets to provide immersive training experiences. These peripherals 710 enable user interaction with the training element 700, allowing for the presentation of visual and auditory stimuli and the capture of user responses during training tasks. The peripherals 710 may serve as the primary interface for implementing gamified cognitive training exercises and computerized tasks.

A network interface 715 is included in the training element 700 to facilitate communication with external systems and services. The network interface 715 may support various connectivity options such as Wi-Fi, cellular data, or Ethernet, enabling the training element 700 to transmit and receive data, access cloud-based resources, and integrate with larger cognitive assessment and rehabilitation systems.

The training element 700 contains memory 720, which may include both volatile and non-volatile storage components. The memory 720 stores the operating system, application software, and data necessary for the functioning of the training element 700. In some cases, the memory 720 may also cache training tasks and user data to improve performance and reduce network dependency.

A training application 725 resides within the memory 720 of the training element 700. The training application 725 may contain the logic and processes necessary to deliver cognitive training tasks, process user responses, and adapt difficulty levels based on performance. In some cases, the training application 725 may be modular, allowing for easy updates and customization of training protocols.

The training element 700 maintains user data 730 within its memory. The user data 730 may include historical performance records, demographic information, and other relevant data specific to individual users. This data may be used to personalize training tasks and track cognitive changes over time.

Model data 735 is also stored within the training element 700. The model data 735 may contain parameters and weights for computational models used in analyzing training performance and adapting difficulty levels. In some cases, the model data 735 may be updated regularly to incorporate the latest research findings and improve training effectiveness.

Various elements for a cognitive training element are discussed above with reference to FIG. 7. Alternative elements may be utilized as appropriate to the requirements of specific applications. These alternative elements also perform cognitive training. These alternatives may provide accurate and robust training and rehabilitation of cognitive abilities in accordance with various embodiments of the invention.

Cognitive training applications provide modular software architectures for delivering personalized cognitive rehabilitation exercises and tracking progress over time. These applications may be implemented on various computing devices, including personal computers, tablets, and smartphones, offering flexibility in deployment and user interaction. Cognitive training applications typically incorporate specialized modules for delivering training tasks, analyzing performance data, and adapting difficulty levels based on user progress.

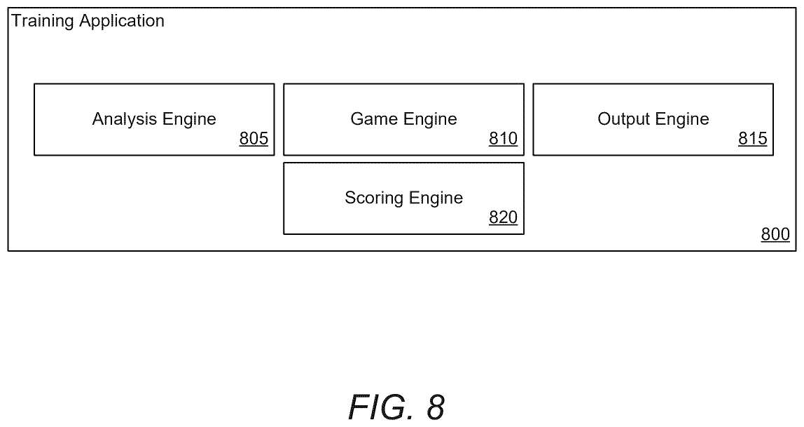

A cognitive training application in accordance with an embodiment of the invention is illustrated in FIG. 8. The training application 800 includes an analysis engine 805 that serves as the core processing unit for cognitive training. The analysis engine 805 may execute processes to analyze user performance data from training tasks, identify patterns in user responses, and generate insights about the user's cognitive strengths and weaknesses. In some cases, the analysis engine 805 may utilize machine learning models to enhance the accuracy of performance analysis and adaptation of training protocols.

A game engine 810 is incorporated within the training application 800. The game engine 810 may be responsible for generating and managing interactive exercises and games for cognitive training. The game engine 810 may utilize insights provided by the analysis engine 805 to dynamically adjust the difficulty and type of tasks presented to the user, ensuring an optimal challenge level for effective cognitive training. In some cases, the game engine 810 may incorporate virtual reality elements, allowing for training experiences with different levels of mobility and physical exertion.

The training application 800 contains an output engine 815 to present training tasks and feedback to the user. The output engine 815 may generate visual and auditory elements of the games, ensuring a clear and engaging interface. In some cases, the output engine 815 may present performance metrics and progress reports, helping users track their improvement over time. The output engine 815 may also be responsible for implementing specialized audiovisual features, such as presenting a 40 Hz flickering visual stimulus and 40 Hz amplitude-modulated sound during training breaks.

A scoring engine 820 is included in the training application 800 to evaluate user performance across various cognitive tasks. The scoring engine 820 may calculate scores based on accuracy, speed, and consistency, providing quantitative measures of cognitive abilities. In some cases, the scoring engine 820 may work closely with the analysis engine 805 to contribute to the overall assessment of the user's cognitive profile and inform the adaptation of training protocols.

The analysis engine 805, game engine 810, output engine 815, and scoring engine 820 work in concert to deliver comprehensive cognitive training experiences. The game engine 810 may generate training tasks, which are then presented to the user through the output engine 815. User responses may be processed by the analysis engine 805 and evaluated by the scoring engine 820. The results of this analysis may then inform the game engine 810's adaptation of task difficulty and type, creating a personalized and dynamic training experience.

In some cases, the training application 800 may incorporate virtual reality training protocols. These protocols may include short intervals of physical activities between cognitive tasks, leveraging the capabilities of virtual reality systems to combine cognitive and physical exercise. The game engine 810 may generate these physical activity prompts, while the output engine 815 presents them within the virtual environment.

Various applications for a cognitive training application are discussed above with reference to FIG. 8. Alternative applications may be utilized as appropriate to the requirements of specific applications. These alternative applications also perform cognitive training and rehabilitation. These alternatives may provide accurate and robust training and rehabilitation of cognitive abilities in accordance with various embodiments of the invention.

A number of implementations have been described. Nevertheless, it will be understood that various modifications may be made without departing from the spirit and scope of the disclosure. Accordingly, other implementations are within the scope of the following claims.

1. A method for cognitive assessment, comprising:
presenting, using an assessment device, one or more tasks to a subject, wherein the one or more tasks include a time series of visual stimuli, audio stimuli, or both visual and audio stimuli, wherein the visual stimuli comprises flashes of light and the audio stimuli comprises short audio tones, wherein the short audio tones comprises noise bursts;
receiving, via the assessment device, response data from the subject for each of the one or more tasks;
calculating, using one or more processors, performance-related parameters based on the response data, wherein the performance-related parameters include timing data;
analyzing, using the one or more processors, the subject's performance as a combination of performance-related parameters selected across the one or more tasks presented to the subject; and
generating, using the one or more processors, a cognitive measurement of the subject based on the analyzed performance.

2. The method of claim 1, wherein the one or more tasks presented to the subject comprise at least one of: a temporal numerosity judgment task, a rhythm discrimination task, a pitch discrimination task, and a simple reaction-time task.

3. The method of claim 2, wherein the temporal numerosity judgment task comprises:
presenting a variable number of visual flashes and auditory tones;
recording the subject's judgment of the number of flashes and tones; and
analyzing an accuracy and precision of the subject's judgments.

4. The method of claim 2, wherein the rhythm discrimination task comprises:
presenting sequences of visual or auditory stimuli with varying time intervals;
recording the subject's responses in comparing, matching, or judging the rhythms; and assessing the subject's accuracy, precision, and/or thresholds in perceiving the rhythms.

5. The method of claim 2, wherein the pitch discrimination task comprises:
presenting a sequence of auditory tones to the subject, wherein each tone has a specific frequency;
instructing the subject to identify a relationship between the frequencies of the tones;
recording the subject's responses and response times for each pair of tones;
adjusting the frequency difference between tones based on the subject's performance to determine a pitch discrimination threshold; and
analyzing the subject's pitch discrimination ability based on the recorded responses, response times, and determined threshold.

6. The method of claim 1, wherein generating the cognitive measurement comprises generating a global score based on the subject's performance across the one or more tasks.

7. The method of claim 1, wherein the one or more tasks utilizes only one sensory modality of either visual and auditory.

8. The method of claim 1, wherein the one or more tasks utilizes a combination of both visual and auditory modality.

9. The method of claim 1, further comprising determining a pattern of performance based on the subject's performance across the one or more tasks.

10. The method of claim 6, wherein the global score is generated by normalizing scores from different types of tasks and calculating a weighted average of the normalized scores.

11. A method for cognitive training, comprising:
determining, using a training device, a training level based on a cognitive measurement of a subject;
presenting, using the training device, one or more tasks to the subject, wherein the one or more tasks include a time series of visual stimuli, audio stimuli, or both visual and audio stimuli for cognitive training based on the determined training level;
collecting, using the training device, responses from the subject in response to the presented stimuli;
calculating, using one or more processors, timing and performance related parameters based on the collected responses;
analyzing, using the one or more processors, the subject's performance based on the calculated timing and performance related parameters;
determining, using the one or more processors, the subject's performance relative to the cognitive training; and
adjusting, using the one or more processors, the training level based on the determined performance.

12. The method of claim 11, wherein adjusting the training level comprises increasing or decreasing a difficulty of the visual task, auditory task, or both sensorimotor task presented to the subject.

13. The method of claim 11, wherein the cognitive training comprises at least one of: a computerized task, and an immersive task.

14. The method of claim 13, wherein the computerized task comprises:
presenting concurrent visual and auditory stimuli to the subject, wherein the stimuli consist of sequences of brief visual and auditory events presented in rapid succession;
instructing the subject to process a temporal structure of the visual and auditory stimuli;
prompting the subject to perform at least one selected from the group consisting of:
determining whether two audiovisual sequences are the same or different;
determining whether the auditory and visual sequences match;
responding with temporal precision to the end of a sequence in one modality cued by another modality; and
providing feedback to the subject on each trial about a correctness and speed of their response.

15. The method of claim 13, wherein the immersive task comprises:
presenting concurrent rhythmic visual and auditory stimuli to the subject in a simulated environment;
instructing the subject to process and respond to the concurrent stimuli with temporal precision;
prompting the subject to perform actions in response to the concurrent stimuli, wherein the actions simulate interaction with virtual objects;
tracking the subject's movements and responses in relation to the presented stimuli;
providing real-time feedback to the subject on their performance; and
adaptively adjusting a difficulty of the task based on the subject's performance.

16. The method of claim 15, wherein the immersive task is delivered through a virtual reality interface.

17. The method of claim 16, wherein the virtual reality interface presents gamified cognitive training exercises that combine cognitive tasks with physical activities, and utilize a musical rhythmic game.

18. The method of claim 11, further comprising presenting a 40 Hz flickering visual stimulus and a 40 Hz amplitude-modulated sound during breaks in the cognitive training.

19. The method of claim 11, wherein calculating timing and performance-related parameters comprises recording accuracy, reaction times, and consistency across multiple training sessions.

20. A cognitive assessment and training system, comprising:
a memory;
one or more processors; and
an input/output device;
wherein the one or more processors are configured to execute instructions stored in the memory to:
present one or more assessment tasks to a subject via the input/output device, wherein the one or more tasks include a time series of visual stimuli, audio stimuli, or both visual and audio stimuli, wherein the visual stimuli comprises flashes of light and the audio stimuli comprises short auditory tones, wherein the short audio tones comprises noise bursts;
receive response data from the subject for each of the one or more tasks via the input/output device;
calculate performance-related parameters based on the response data, wherein the performance-related parameters include timing data;
analyze the subject's performance as a combination of performance-related parameters selected across the one or more tasks presented to the subject; generate a cognitive measurement of the subject based on the analyzed performance;
determine a training level based on the cognitive measurement;
present a time series of visual stimuli, audio stimuli, or both visual and audio stimuli to the subject for cognitive training via the input/output device based on the determined training level;
collect responses from the subject in response to the presented stimuli;
calculate timing and performance related parameters based on the collected responses;
analyze the subject's performance based on the calculated timing and performance related parameters;
determine the subject's performance relative to the cognitive training; and
adjust the training level based on the determined performance.

21. The cognitive assessment and training system of claim 20, wherein the one or more assessment tasks comprise at least one of: a temporal numerosity judgment task, a rhythm discrimination task, a pitch discrimination task, and a simple reaction-time task.

22. The cognitive assessment and training system of claim 21, wherein the temporal numerosity judgment task comprises:
presenting a variable number of visual flashes and auditory tones;
recording the subject's judgment of the number of flashes and tones; and
analyzing an accuracy and precision of the subject's judgments.

23. The cognitive assessment and training system of claim 21, wherein the rhythm discrimination task comprises:
presenting sequences of visual or auditory stimuli with varying time intervals;
recording the subject's responses in comparing, matching, or judging the rhythms; and assessing the subject's accuracy, precision, and/or thresholds in perceiving the rhythms.

24. The cognitive assessment and training system of claim 21, wherein the pitch discrimination task comprises:
presenting a sequence of auditory tones to the subject, wherein each tone has a specific frequency;
instructing the subject to identify a relationship between the frequencies of the tones;
recording the subject's responses and response times for each pair of tones;
adjusting the frequency difference between tones based on the subject's performance to determine a pitch discrimination threshold; and
analyzing the subject's pitch discrimination ability based on the recorded responses, response times, and determined threshold.

25. The cognitive assessment and training system of claim 21, wherein the cognitive training comprises at least one of: a computerized task, and an immersive task.

26. The cognitive assessment and training system of claim 25, wherein the computerized task comprises:
presenting concurrent visual and auditory stimuli to the subject, wherein the stimuli consist of sequences of brief visual and auditory events presented in rapid succession;
instructing the subject to process a temporal structure of the visual and auditory stimuli;
prompting the subject to perform at least one selected from the group consisting of:
determining whether two audiovisual sequences are the same or different;
determining whether the auditory and visual sequences match;
responding with temporal precision to the end of a sequence in one modality cued by another modality; and
providing feedback to the subject on each trial about a correctness and speed of their response.

27. The cognitive assessment and training system of claim 25, wherein the immersive task comprises:
presenting concurrent rhythmic visual and auditory stimuli to the subject in a simulated environment;
instructing the subject to process and respond to the concurrent stimuli with temporal precision;
prompting the subject to perform actions in response to the concurrent stimuli, wherein the actions simulate interaction with virtual objects;
tracking the subject's movements and responses in relation to the presented stimuli;
providing real-time feedback to the subject on their performance; and
adaptively adjusting a difficulty of the task based on the subject's performance.

28. The cognitive assessment and training system of claim 20, wherein generating the cognitive measurement comprises generating a global score based on the subject's performance across the one or more tasks.

29. The cognitive assessment and training system of claim 28, wherein the global score is generated by normalizing scores from different types of tasks and calculating a weighted average of the normalized scores.

30. The cognitive assessment and training system of claim 20, wherein the one or more processors are further configured to present a 40 Hz light flickering visual stimulus and a 40 Hz amplitude-modulated sound during breaks in the cognitive training.
